(19) United States
(12) Patent Application Publication
(10) Pub. No.: US 2019/0300843 A1
(43) Pub. Date: Oct. 3, 2019

(54) METHOD FOR ARCHIVING A MICROBIOME

(71) Applicant: Kimberly-Clark Worldwide, Inc., Neenah, WI (US)

(72) Inventors: Rebecca A. Vongsa, Neenah, WI (US); David W. Koenig, Menasha, WI (US)

(21) Appl. No.: 16/346,201

(22) PCT Filed: Oct. 25, 2017
(86) PCT No.: PCT/US17/58182

§ 371 Date: Apr. 30, 2019

Related U.S. Application Data
(60) Provisional application No. 62/414,937, filed on Oct. 31, 2016.

(51) Int. Cl. C12N 1/20; A61K 35/741; C12N 1/04; C12N 1/36; A23L 33/135

(52) U.S. Cl. CPC C12N 1/20; A61K 35/741; A23L 33/135; C12N 1/36; C12N 1/04

(57) ABSTRACT

Methods for archiving and reconstituting a microbiome are disclosed. A method for archiving a microbiome can include collecting a sample including a microbial community defining the microbiome. The method can also include expanding the microbial community in the sample and preserving the sample to provide a preserved sample. Packaging the preserved sample to archive the microbiome can also form part of the method of archiving the microbiome.

### CROSS-REFERENCE TO RELATED APPLICATIONS

This application claims priority to U.S. Provisional Patent Application No. 62/414,937 filed on Oct. 31, 2016, which is incorporated herein by reference in its entirety for all purposes.

### BACKGROUND

The present disclosure relates to a method for archiving a microbiome. More specifically, the present disclosure relates to a method for archiving a human microbiome.

The human body is covered in microbial communities that have adapted to occupy a certain niche. For example, the human body includes microbial communities such as the skin, vaginal canal, intestine, and other mucosal membranes.

There are a variety of reasons a person may want to archive a personal, living microbiome of one of these microbial communities from a healthy time of their life. For instance, transferring the maternal microbiome to a baby may present one situation in which a personal, living microbiome can be reconstituted and utilized to treat a particular condition. Another example for archiving a human microbiome can be to use as a personal probiotic in association with antibiotic use. An intestinal microbiome could be archived and used in a fecal transplant to treat certain diseases and/or conditions of the digestive track. Furthermore, restoration of a desirable microbial community could be accomplished after antibiotic therapy, chemotherapy, radiation, or other events taking a toll on specific microbial communities of the body.

However, consumer friendly options to archive a personal microbiome are limited. As such, it is desirable to provide a method for archiving a microbiome. It is also desirable to provide a method for archiving a microbiome that is stable and would allow for easy reconstitution by a consumer.

### SUMMARY

In one aspect, a method for archiving a microbiome is provided. The method can include collecting a sample including a microbial community defining the microbiome; expanding the microbial community in the sample; preserving the sample to provide a preserved sample; and packaging the preserved sample to archive the microbiome.

In another aspect, a method for archiving a microbiome can include collecting a sample including a microbial community defining the microbiome; providing a microbiome modulator to the sample; preserving the sample to provide a preserved sample; and packaging the preserved sample to archive the microbiome.

In yet another aspect, a method for archiving a microbiome can include collecting a sample including a microbial community defining the microbiome, the microbial community being selected from the group consisting of: nasal, vaginal, skin, oral, bladder, placenta, breast, scalp, ear, eye, kidney, lungs, and nails. The method can also include preserving the sample by lyophilization to provide a preserved sample. The method can additionally include packaging the preserved sample to archive the microbiome.

### DESCRIPTION OF THE DRAWINGS

FIG. 1 illustrates a perspective view of an exemplary kit that can be used in some embodiments of the present disclosure.

FIG. 2 illustrates a top plan view of a dual stage ampoule configured for implementing an aspect of the present disclosure.

FIG. 3 illustrates a cross-sectional view taken along line 3-3 from FIG. 2 depicting a preserved sample of a microbial community in a first chamber and a reconstitution fluid in a second chamber.

### DETAILED DESCRIPTION

The present disclosure relates generally to methods of archiving a microbiome. In some embodiments, the microbiome can be reconstituted in preparation for implementing to a patient or consumer for helping restore a desired microbial community. For purposes of this disclosure, reference to “patient” or “consumer” or “user” can be replaceable, as the way an intended user of the method of the present application is referred to due to a particular condition or reason for restoring a microbiome is not meant to limit in any aspects of the disclosure.

As mentioned above, there are a variety of reasons why a user may want to archive a microbiome. Although some preferred embodiments of the present disclosure relate to archiving a human microbiome, it can be appreciated that the present disclosure is not limited to any specific listing of human microbiomes. Furthermore, it is to be understood that the present disclosure encompasses methods and uses for archiving microbiomes for a variety of other animal and plant species as well as environmental conditions, in addition to human microbiomes. For example, there may be particular interest in archiving one or more microbiomes for pets. Archiving a pet microbiome can be done to reconstitute the microbiome after antibiotic therapy, or possibly, to help restore the microbiome to a more youthful state to help with skin condition or odor problems.

In one embodiment, a method for archiving a microbiome can include collecting a sample including a microbial community defining the microbiome. Collecting a sample can be achieved in various methods depending on the site the microbial sample is intended to be collected. As an example, an intestinal microbial community defining the microbiome of a patient's intestine can be collected via a feces sample (e.g., a stool sample) in a standard fecal collection vial, as is known in the art. For vaginal, skin, nasal, and other body parts, standard microbial specimen swabs can be used. Other collection mechanisms/methods that can be used can include: washes and collection with wipes, vacuum, use of dry absorbents (SAM), and particles (e.g., clay, vermiculite, charcoal). Any suitable collection method or means of collecting a sample including a microbial community defining the microbiome is within the scope of this disclosure.

Upon collecting a sample including the microbial community defining the microbiome, the sample can be temporarily stored to preserve the integrity of the sample. For example, in some embodiments, the sample can be stored in a sealed bag or vial and placed in a standard refrigerator or freezer. Potential storage techniques can include the use of desiccants, osmolites, and chilling with endothermic substrates. It is to be understood that one of ordinary skill in the art understands the appropriate collection equipment and methodology for collecting a sample that will include a microbial community representative of and defining the intended microbiome, as well as proper storage techniques for preserving the integrity of the sample. In some embodiments, the microbial community can be collected in a user's home. In other embodiments, the microbial community can be collected at a medical facility or professional facility. However, the location of where or how the microbial sample is collected is not meant to limit the present disclosure.

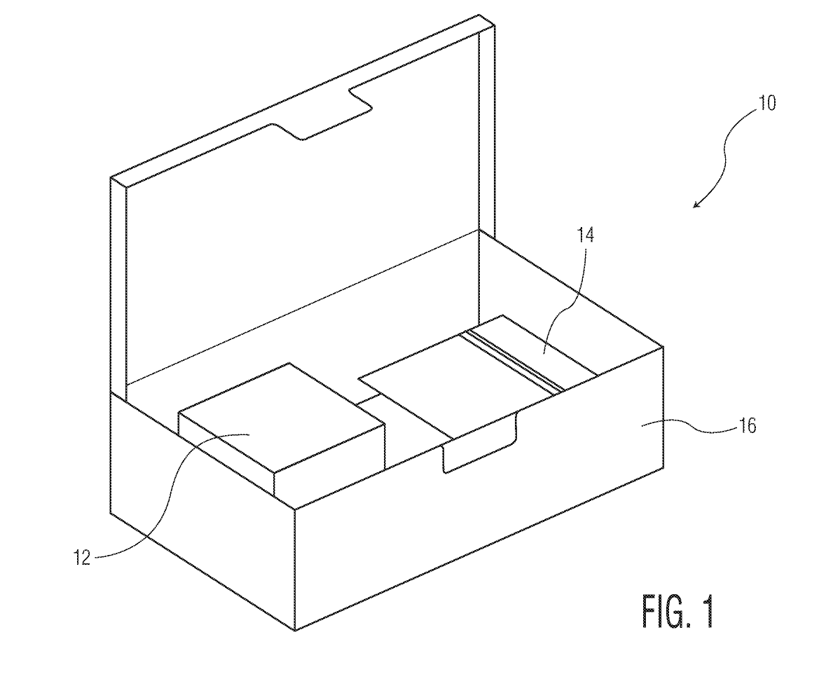

In some embodiments, the sample can be transported to a professional facility for further processing and archiving. For example, if the sample is collected at a user's home, the sample can be transported to a facility for further processing and archiving. In such situations, a user can be provided with a kit 10 that includes the appropriate collection and temporary storage equipment to preserve the integrity of the sample before transporting the sample to a professional facility. As one example, FIG. 1 illustrates a sample kit 10 that could be used for body sites such as the nasal microbiome or skin microbiome. The kit 10 can include a microbial specimen swab 12 for collecting a sample from the user's nose and a sealed bag 14 to temporarily store the swab after the user takes the sample including the microbial community defining the intended microbiome. The kit 10 can also include a container 16 that envelopes the swab 12 and bag 14, and in which the collected sample on the swab 12 and held in the sealed bag 14 could be shipped to a professional facility. Of course, it is contemplated that the method need not involve transporting the sample including the microbiome community defining the microbiome to a professional facility. For example, in some embodiments, the sample may be collected at a professional facility itself. In other embodiments, the sample may be collected at a user's home and remain there for archiving purposes.

Additionally, the method for archiving a microbiome can also include preserving the sample including the microbial community defining the microbiome after the sample is collected to provide a preserved sample. In a preferred embodiment, the sample can be preserved through lyophilization. Lyophilization is a freeze drying process in which water can be removed from the sample after it is frozen and placed under a vacuum. Lyophilization techniques for preserving microbial samples are known by one of ordinary skill in the art. A benefit of lyophilization is that it allows the sample to be stored for long periods of time without the use of cryogenic fluid or freezing the samples in liquid. Lyophilization provides greater dryness than the use of desiccants alone. However, it is contemplated in this disclosure that desiccants can also be utilized to reduce moisture.

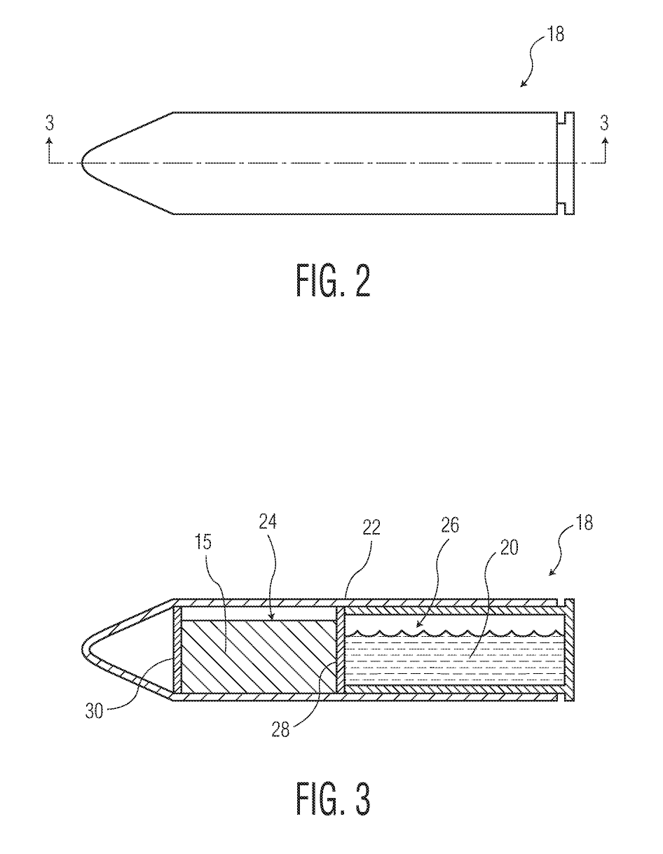

The preserved sample can also be packaged to archive the microbiome. The preserved sample can be packaged in a variety of containers, including, but not limited to, vials, bottles, tubes, bags, receptacles, and ampoules. In a preferred embodiment, the preserved sample can be packaged in a dual chambered ampoule 18, as shown in FIGS. 2 and 3, and as discussed further below. Once packaged, the preserved sample can be archived. In some embodiments, the packaged and preserved sample can be archived by sending to the user for storage in their home, at a professional facility where the sample is collected, or at another storage facility. In some examples, the preserved sample can remain viable for at least 10-50 years and can be archived at room temperature or refrigerated when packaged after lyophilization. As such, the preserved sample can be kept in packaging, such as in a dual chambered ampoule 18 in the convenience of a user's own home. If the sample is stored in a refrigerator or freezer the shelf life can be further extended. Furthermore, adding additional components to the sample such as osmolites (e.g., sorbitol, mannitol, glycerol) can help in stability.

In some preferred embodiments, the method for archiving a microbiome can include expanding the microbial sample that defines the microbiome. Expanding the microbial sample can be accomplished by introducing a microbiome modulator to the sample. A microbiome modulator can expand the microbial community by encouraging growth of a desired bacteria in the microbial community. In some embodiments, the microbiome modulator could comprise a prebiotic. For instance, for the vaginal microbiome, a media such as LAPTg may be used that has been demonstrated to support growth of lactobacilli and other Gram negative and Gram positive organisms typically found in the vagina. The expansion can be done in low oxygen conditions (e.g., about 2%), which mimic conditions in the vagina. Addition of microbiome modulators like the prebiotic inulin could be used to preferably grow Lactobacillus spp that are known to be beneficial in the vaginal canal. Prebiotics that could be used to encourage growth of Lactobaccilus spp include saccharides that may not be digestible by humans but are useful prebiotics within the scope of the present disclosure and can include, but are not limited to, transgalactooligosaccharides, galacto-oligosaccharides, lactulose, raffinose, stachyose, lactosucrose, fructo-oligosaccharides, isomalto-oligosaccharides, xylo-oligosaccharides, paratinose oligosaccharides, difructose anhydride III, sorbitol, maltitol, lactitol, reduced paratinose, cellulose, β-glucose, β-galactose, β-fructose, verbascose, galactinol, and β-glucan, guar gum, pectin, high sodium alginate, and lambda carrageenan.

It is preferable to expand the microbial sample prior to preserving the sample, however, it is contemplated that the microbial sample could be expanded after the sample is preserved and archived as part of reconstituting the microbiome, as will be further discussed below. It is also contemplated that the microbial sample can be expanded both prior to preserving the sample and after preserving the microbial sample during reconstituting the microbiome.

Additionally or alternatively, the microbiome modulator can also discourage growth of a non-desired bacteria in the microbial community of the sample. For example, a microbiome modulator such as xylitol may be used to discourage the growth of Streptococcus in the oral microbiome. Additionally, the microbiome modulator can include a bacteriophage and/or a predatory bacteria that discourage growth of a non-desired bacteria in the microbial community of the sample. One example of a predatory bacteria could include Bdellovibrio bacteriovorus that are known to prey on Gram negative pathogens. Exemplary bacteriophage that can discourage growth of non-desired bacteria can include Staphylococcal Myoviridae (e.g. phages K, G1, Twort), Pseudomonas PAK-P1 bacteriophage, and Enterobacteria phage T4. Such bacteriophage and/or predatory bacteria could be useful for discouraging growth of non-desired bacteria in a variety of microbial communities, including, but not limited to, the skin, nasal, scalp, perianal, and toe. Of course, other bacteriophage and predatory bacteria that discourage growth of a non-desired bacteria in the microbial community of the sample are within the scope of this disclosure.

As discussed above, it can be preferable to discourage growth of a non-desired bacteria prior to preserving the sample, however, it is contemplated that the microbial sample could be provided with a microbiome modulator that discourages growth of a non-desired bacteria after the sample is preserved and archived as part of reconstituting the microbiome, as will be further discussed below. It is also contemplated that the microbial sample can be provided with a microbiome modulator that discourages growth of a non-desired bacteria both prior to preserving the sample and after preserving the microbial sample during reconstituting the microbiome.

As illustrated in FIGS. 2 and 3, and as alluded to above, an exemplary preserved sample 15 including the microbial community defining the microbiome can be packaged such that a reconstitution fluid 20 is provided along with the preserved sample 15, but the reconstitution fluid 20 is kept separate from the preserved sample 15. Examples of reconstitution fluids include LAPTg, buffer saline, defined and undefined media, or water. In some embodiments, a preferred reconstitution fluid can include minimal salts and can include osmolites. The reconstitution fluid can also include microbiome modulators in some embodiments, for example, a prebiotic.

As depicted in FIGS. 2 and 3, a dual chambered ampoule 18 can be employed for archiving the preserved sample 15 along with a reconstitution fluid 20 in this fashion. The dual chambered ampoule 18 can include a housing 22 that provides a first chamber 24 and a second chamber 26. The exemplary preserved sample 15 including the microbial community defining the microbiome can be housed within the first chamber 24. A reconstitution fluid 20 can be provided in the second chamber 26 of the ampoule 18. As noted above, in some embodiments, the exemplary preserved sample 15 can be modulated by a microbiome modulator (either by growth of the sample, or discouraging of growth of a non-desired bacteria) prior to preserving the exemplary sample 15 and it being inserted into the ampoule 18. Alternatively or additionally, a microbiome modulator can be provided within the reconstitution fluid 20 such that the microbiome modulator could modulate the exemplary sample 15 when the sample 15 is desired to be reconstituted.

The preserved sample including the microbial community defining the microbiome can be reconstituted after the sample is archived. As noted above, some preservation methods, such as lyophilization, may allow for the preserved sample to be archived and remain stable for upwards of 50 years, even in a user's home. In the embodiment depicted in FIGS. 2 and 3, the dual chambered ampoule 18 can provide benefits for reconstituting the microbiome for a user. To reconstitute the exemplary preserved sample 15, once a user desires to reconstitute the exemplary preserved sample, a seal 28 separating the first chamber 24 from the second chamber 26 can be broken to reconstitute the preserved sample 15 and recreate the microbiome. A further seal 30 can then be broken to apply the reconstituted sample to the desired application. Of course, it is to be understood that a dual chamber ampoule 18 is just one device that can be used and helpful for reconstituting the microbiome, and the present disclosure is meant to cover any other known device that could be used to reconstitute the sample of the microbial community that would be sufficient. While preferred, the device for storing the archived sample need not be the same device in which the sample is reconstituted.

The dual chambered ampoule 18 can form part of the kit 10 discussed above and depicted in FIG. 1. In some embodiments, a kit 10 can be configured to include the dual chambered ampoule 18 which can be transported to a professional facility after the sample including the microbial community is collected in the microbial specimen swab 12 and temporarily stored in the sealed bag 14. After the facility preserves the sample and places the preserved sample in the dual chambered ampoule 18, the dual chambered ampoule 18 can be archived by the user at their location of choice. As noted above, the dual chambered ampoule 18 could be archived at the user's residence and be ready for reconstitution at the user's choice or the dual chambered ampoule 18 could be archived at a professional facility until requested by the user. Reconstituting the sample 15 could be accomplished by the user by mixing the reconstitution fluid 20 with the sample 15, as discussed above. Such a kit 10 can provide the benefits of each of use for a user to collect, preserve, archive, and reconstitute a microbiome.

Archiving a microbiome can have a wide variety of applications within the human body. For example, microbiomes that a user may desire to archive for potential benefit at a later time can include, but are not limited to: gut/intestinal, nasal, vaginal, skin, oral, bladder, placenta, breast, scalp, ear, eye, kidney, lungs, and nails. A user may wish to reconstitute and use such a microbiome for maternal transfer or vaginal seeding for C-sections or for fecal transplants. Other potential reasons for reconstituting and using such microbiomes could be for cosmetic reasons, such as seeding the skin with a younger microbiome. Additionally, specific species within a microbial community may be restored that may have been lost to some events such as antibiotic therapy, chemotherapy, radiation, or other events. There may also be similar uses for species other than humans, for example, particularly with pets. Additional benefits could be for livestock such as dairy cows, horses, beef cattle, pigs, and chickens for increased production and health.

### EMBODIMENTS

Embodiment 1: A method for archiving a microbiome, the method comprising: collecting a sample including a microbial community defining the microbiome; expanding the microbial community in the sample; preserving the sample to provide a preserved sample; and packaging the preserved sample to archive the microbiome.

Embodiment 2: The method of embodiment 1, wherein expanding the microbial community in the sample includes providing a microbiome modulator to the sample.

Embodiment 3: The method of embodiment 2, wherein providing the microbiome modulator to the sample occurs prior to preserving the sample.

Embodiment 4: The method of embodiment 2 or embodiment 3, wherein the microbiome modulator comprises a prebiotic that encourages growth of a desired bacteria in the microbial community.

Embodiment 5: The method of embodiment 4, wherein the microbiome modulator further comprises at least one of a bacteriophage and a predatory bacteria that discourage growth of a non-desired bacteria in the microbial community.

Embodiment 6: The method of any one of the preceding embodiments, wherein the sample is preserved by lyophilization.

Embodiment 7: The method of any one of the preceding embodiments, wherein packaging the preserved sample includes providing a reconstitution fluid along with the preserved sample, wherein the reconstitution fluid is separate from the preserved sample.

Embodiment 8: The method of embodiment 7, wherein the microbiome modulator is provided in the reconstitution fluid.

Embodiment 9: The method of embodiment 7 or embodiment 8, wherein the packaging is a dual chambered ampoule including a first chamber and a second chamber, the first chamber of the ampoule includes the preserved sample, the second chamber of the ampoule includes the reconstitution fluid.

Embodiment 10: A method for archiving a microbiome, the method comprising: collecting a sample including a microbial community defining the microbiome; providing a microbiome modulator to the sample;

preserving the sample to provide a preserved sample; and packaging the preserved sample to archive the microbiome.

Embodiment 11: The method of embodiment 10, wherein providing the microbiome modulator to the sample occurs prior to preserving the sample.

Embodiment 12: The method of embodiment 10 or embodiment 11, wherein the microbiome modulator comprises a prebiotic that encourages growth of a desired bacteria in the microbial community.

Embodiment 13: The method of any one of embodiments 10-12, wherein the microbiome modulator comprises at least one of a bacteriophage and a predatory bacteria that discourage growth of a non-desired bacteria in the microbial community.

Embodiment 14: The method of any one of embodiments 10-13, wherein the sample is preserved by lyophilization.

Embodiment 15: The method of any one of embodiments 10-14, wherein packaging the preserved sample includes providing a reconstitution fluid along with the preserved sample, wherein the reconstitution fluid is separate from the preserved sample.

Embodiment 16: The method of embodiment 15, wherein the microbiome modulator is provided in the reconstitution fluid.

Embodiment 17: The method of embodiment 15 or 16, wherein the packaging is a dual chambered ampoule including a first chamber and a second chamber, the first chamber of the ampoule includes the preserved sample, the second chamber of the ampoule includes the reconstitution fluid.

Embodiment 18: A method of recreating a microbiome, the method comprising: archiving the microbiome according to the method for archiving a microbiome of any of the preceding embodiments; and reconstituting the preserved sample to recreate the microbiome.

Embodiment 19: A method for archiving a microbiome, the method comprising: collecting a sample including a microbial community defining the microbiome, the microbial community being selected from the group consisting of: nasal, vaginal, skin, oral, bladder, placenta, breast, scalp, ear, eye, kidney, lungs, and nails; preserving the sample by lyophilization to provide a preserved sample; and packaging the preserved sample to archive the microbiome.

Embodiment 20: The method of embodiment 19, further comprising: providing a microbiome modulator to the sample, the microbiome modulator comprising at least one of a prebiotic, a bacteriophage, and a predatory bacteria, wherein the prebiotic is configured to encourage growth of a desired bacteria in the microbial community, and wherein the bacteriophage and the predatory bacteria are configured to discourage growth of a non-desired bacteria in the microbial community.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

What is claimed is:

1. A method for archiving a microbiome, the method comprising:
collecting a sample including a microbial community defining the microbiome;
expanding the microbial community in the sample;
preserving the sample to provide a preserved sample; and
packaging the preserved sample to archive the microbiome.

2. The method of claim 1, wherein expanding the microbial community in the sample includes providing a microbiome modulator to the sample.

3. The method of claim 2, wherein providing the microbiome modulator to the sample occurs prior to preserving the sample.

4. The method of claim 2, wherein the microbiome modulator comprises a prebiotic that encourages growth of a desired bacteria in the microbial community.

5. The method of claim 4, wherein the microbiome modulator further comprises at least one of a bacteriophage and a predatory bacteria that discourage growth of a non-desired bacteria in the microbial community.

6. The method of claim 1, wherein the sample is preserved by lyophilization.

7. The method of claim 2, wherein packaging the preserved sample includes providing a reconstitution fluid along with the preserved sample, wherein the reconstitution fluid is separate from the preserved sample.

8. The method of claim 7, wherein the microbiome modulator is provided in the reconstitution fluid.

9. The method of claim 7, wherein the packaging is a dual chambered ampoule including a first chamber and a second chamber, the first chamber of the ampoule includes the preserved sample, the second chamber of the ampoule includes the reconstitution fluid.

10. A method for archiving a microbiome, the method comprising:
collecting a sample including a microbial community defining the microbiome;
providing a microbiome modulator to the sample;
preserving the sample to provide a preserved sample; and
packaging the preserved sample to archive the microbiome.

11. The method of claim 10, wherein providing the microbiome modulator to the sample occurs prior to preserving the sample.

12. The method of claim 10, wherein the microbiome modulator comprises a prebiotic that encourages growth of a desired bacteria in the microbial community.

13. The method of claim 10, wherein the microbiome modulator comprises at least one of a bacteriophage and a predatory bacteria that discourage growth of a non-desired bacteria in the microbial community.

14. The method of claim 10, wherein the sample is preserved by lyophilization.

15. The method of claim 10, wherein packaging the preserved sample includes providing a reconstitution fluid along with the preserved sample, wherein the reconstitution fluid is separate from the preserved sample.

16. The method of claim 15, wherein the microbiome modulator is provided in the reconstitution fluid.

17. The method of claim 15, wherein the packaging is a dual chambered ampoule including a first chamber and a second chamber, the first chamber of the ampoule includes the preserved sample, the second chamber of the ampoule includes the reconstitution fluid.

18. A method of recreating a microbiome, the method comprising:
archiving the microbiome according to the method for archiving a microbiome of claim 10; and
reconstituting the preserved sample to recreate the microbiome.

19. A method for archiving a microbiome, the method comprising:
collecting a sample including a microbial community defining the microbiome, the microbial community being selected from the group consisting of: nasal, vaginal, skin, oral, bladder, placenta, breast, scalp, ear, eye, kidney, lungs, and nails;
preserving the sample by lyophilization to provide a preserved sample; and
packaging the preserved sample to archive the microbiome.

20. The method of claim 19, further comprising:
providing a microbiome modulator to the sample, the microbiome modulator comprising at least one of a prebiotic, a bacteriophage, and a predatory bacteria, wherein the prebiotic is configured to encourage growth of a desired bacteria in the microbial community, and wherein the bacteriophage and the predatory bacteria are configured to discourage growth of a non-desired bacteria in the microbial community.
